(19) United States
(12) Patent Application Publication
(10) Pub. No.: US 2026/0258455 A1
(43) Pub. Date: Sep. 3, 2026

(54) UNIQUE METHOD TO TRIGGER LACTIC ACID PRODUCTION IN STREPTOMYCES COELICOLOR A3(2)

(71) Applicant: GEBZE TEKNIK UNIVERSITESI, Kocaeli (TR)

(72) Inventors: Nagihan GENEL, Kocaeli (TR); Sedef TUNCA GEDIK, Kocaeli (TR)

(73) Assignee: GEBZE TEKNIK UNIVERSITESI, Kocaeli (TR)

(21) Appl. No.: 18/871,504

(22) PCT Filed: Sep. 12, 2023
(86) PCT No.: PCT/TR2023/050945

§ 371 Date: Dec. 4, 2024

(51) Int. Cl. C12P 7/56; C12N 1/20; C12N 15/70; C12Q 1/686; C12R 1/19; C12R 1/465

(52) U.S. Cl. CPC C12P 7/56; C12N 1/20; C12N 15/70; C12Q 1/686; C12R 2001/19; C12R 2001/465

(57) ABSTRACT

A method to trigger lactic acid production in Streptomyces coelicolor A3(2) is provided. In the present invention, the morphology and metabolism of the recombinant Streptomyces coelicolor A3(2) cell totally changed because of using “aadA gene” as a selective marker. After various experiments with different controls it was realized that this change is coming only from the expression of “aadA gene” itself.

### CROSS REFERENCE TO THE RELATED APPLICATIONS

This application is the national phase entry of International Application No. PCT/TR2023/050945, filed on Sep. 12, 2023, the entire contents of which are incorporated herein by reference.

### TECHNICAL FIELD

The invention relates a method to trigger lactic acid production in Streptomyces coelicolor A3(2).

### BACKGROUND

Lactic acid is an organic compound produced via fermentation by different microorganisms that are able to use different carbohydrate sources. Lactic acid bacteria are the main bacteria used to produce lactic acid and among these, Lactobacillus spp. have been showing interesting fermentation capacities. “PCR-targeted Streptomyces gene replacement identifies a protein domain needed for biosynthesis of the sesquiterpene soil odor geosmin” titled publication discloses an efficient procedure for creating precise gene replacements in the cosmid clones by using PCR targeting and λ-Red-mediated recombination. “Intergeneric Conjugation in Streptomyces peucetius and Streptomyces sp. strain C5: Chromosomal Integration and Expression of Recombinant Plasmids Carrying the chiC Gene” titled publication discloses Intergeneric Conjugation in Streptomyces peucetius and Streptomyces sp. strain C5.

U.S. Pat. No. 10,190,139B2 discloses tools and methods for producing organic acids using strains of Monascus which are tolerant to high organic acid concentrations at low pH.

CN103396974A discloses a material and a method for efficient lactic acid production. In this document, a study targeting the inactivation of competitive pathways to increase % lactic acid production in E. coli strains was reported. This application may not give the expected result in Streptomyces spp. because the metabolic network of these microorganisms is quite complex compared to E. coli cells. In addition, to enhance production of a metabolite, first of all these metabolites has to be produced by the organism. To the best of our knowledge, there is no study that draws attention to the lactic acid production by Streptomyces. Moreover, for enhancement of any metabolite production, a dozen of competing pathways must be targeted in this microorganism and this is a time and material consuming process.

### SUMMARY

The object of the present invention is to develop a simple method to trigger lactic acid production in Streptomyces coelicolor A3(2).

This method includes the expression of an antibiotic resistance gene (aadA) which alters cell's metabolism, that is activating primary metabolite pathway (hetero lactic fermentation pathway) ceasing secondary metabolite pathway.

### DETAILED DESCRIPTION OF THE EMBODIMENTS

A method to trigger lactic acid production in Streptomyces coelicolor A3(2) (SCO), the method including:
Amplifying the Streptomycin resistance gene (aadA) by polymerase chain reaction (PCR) using the pIJ778 plasmid as the template,
Cloning the aadA gene into the pRA plasmid, which is an integrative and conjugative vector derived from pSET152 vector.
Transferring the recombinant pRA^{str} plasmid into Escherichia coli ET12567 /pUZ8002 strain.
Performing the conjugation between Streptomyces coelicolor A3(2) and E. coli ET12567/pUZ8002+pRA^{str}
Obtaining new recombinant cell (SCO+pRA^{str}).

In the present invention, first, the aadA gene was amplified by PCR using the pIJ778 plasmid as the template. Then, the aadA gene was cloned into the integrative pRA plasmid. The new recombinant plasmid, pRA^{str}, was transferred into Escherichia coli ET12567 /pUZ8002 strain. Conjugation was performed between S. coelicolor A3(2) and E. coli ET12567 /pUZ8002+ pRA^{str}. New recombinant cell (SCO+pRA^{str}) was obtained according to their streptomycin resistance. Southern Blot hybridization is done to proove the recombinant cell. Lactic acid production by SCO+pRA^{str} was detected by using high liquid pressure chromatography (HPLC). Expression of this resistance gene triggers lactic acid production in Streptomyces coelicolor A3(2) by shifting the carbon flow to lactic acid production instead of antibiotic production. Streptomyces coelicolor A3(2) is original wild type strain that gained lactic acid production ability after expressing streptomycin resistance gene (aadA).

In the present invention, the morphology of the recombinant Streptomyces coelicolor A3(2) cell totally changed because of the using of “aadA gene” as a selective marker. After various experiments with different controls it was realized that the problem is coming only from the expression of “aadA gene” itself. To proove this, pIJ778 plasmid was used just as the DNA template in order to amplify aadA gene by PCR. Then aadA gene was cloned into two different plasmids (integrative pRA plasmid and multicopy pSPG plasmid). Later, the two recombinant plasmids (pRA^{str}, pSPG^{str}) and pIJ 778, which were inside methylation deficient E. coli ET12567(pUZ8002) strain, were transferred into S. coelicolor by RP4 mediated intergeneric conjugation and it was determined that the expression of aadA gene not only affected the morphology but also the metabolism of S. coelicolor A3(2). Recombinant cells expressing aadA gene SCO+pRA^{str}, SCO+pSPG^{str}, SCO+pIJ778, stop producing colored antibiotics and surprisingly start to produce lactic acid and acetate which are important primary metabolites.

HPLC results of 48 h samples of SCO+pRA^{str} strain was found to be 8.9 g/L lactic acid and 3.52 g/L acetic acid. HPLC measurements with 48 h samples showed that SCO+pSPG^{str} was the highest (10.1 g/L) and SCO+pIJ778 was the lowest (7.6 g/L) producer of lactic acid. In the production of acetic acid, SCO+pRA^{str} (3.52 g/L) took first place and continued as SCO+pSPG^{str} (3.08 g/L) and SCO+pIJ778 (2.88 g/L). The results, strongly support that aadA gene expression unexpectedly triggers the heterolactic fermentation pathway in S. coelicolor A3(2) cells.

Heterolactic fermentation is the process of converting glucose into ethanol, lactic acid, and carbon dioxide. According to BioCyc data, most pathway related genes have not yet been identified in Streptomyces and no studies have been conducted on the fermentation pathway of Streptomyces. The present invention is the first one which shows that this pathway is present in Streptomyces and can be activated.

In addition to the present invention, other Streptomyces strains like S. clavuligerus (SCLA) and S. lividans (SLI) were transformed with pRA^{str} plasmid, in order not to be restricted in the framework of S. coelicolor cells. The results showed that the colony morphology of the recombinant SLI+pRA^{str} and SCLA+pRA^{str} strains was similar to SCO+pRA^{str} and SCO+pSPG^{str} strains and they also gained the ability to produce lactic acid and acetic acid. The first 48 h measurement results are as follows; 0.41 g/L lactic acid and 1.25 g/L acetic acid for SLI+pRA^{str}, 1.78 g/L lactic acid and 0.2 g/L acetic acid for SCLA+pRA^{str}. In short, the aadA gene expression unexpectedly changed not only the morphology but also the metabolism of the Streptomyces.

The integrative pRA plasmid, that we used to clone aadA gene, derived from pSET152 plasmid and can integrate itself into the chromosome via the attB regions using the oc31 integrator-mediated system. When the aadA gene is integrated into the genome, surprisingly the metabolism of the bacterium completely changes and result in activation of heterolactic fermentation pathway in S. coelicolor A3(2), S. clavuligerus and S. lividans. This result was unexpected since up to now there is no information about any “antibiotic resistance gene” that modulate the metabolism of a cell. All the antibiotic resistance genes used in genetic engineering studies are effective to select putative transformants/conjugants without changing the metabolism, however we found that this is not true for aadA. By the expression of this gene, S. coelicolor, S. clavuligerus and S. lividans became lactic acid producers instead of antibiotic producers.

Although metabolic changes in bacteria are mostly achieved by complex metabolic engineering strategies, this invention enables non-lactic acid producer S. coelicolor A3(2) wild type cells to become a stable producer of lactic acid with a very simple method. Lactic acid is a very important metabolite in industry and from 2023 to 2030 the global lactic acid market is projected to grow at a compound annual growth rate (CAGR) of 8.0%.

The invention has potential to be applied to industry. First of all, the recombinant strain is genetically stable, that is, aadA gene is integrated into the genome and its expression can be achieved without streptomycin in the medium. According to the unoptimized current data, the recombinant cell (SCO+pRA^{str}) can produce 8.9 g/L lactic acid and 3.52 g/L acetic acid in standard R2 yeast extract (R 2YE) liquid culture within 48 hours. Moreover, it was determined that the growth rate of the recombinant cell increased significantly compared to the wild type; it has the potential to grow in liquid and solid R 2YE culture within 24 hours, making the recombinant Streptomyces strain an important candidate for industry since the time required for seed culture preparation and lactic acid production become shorter. Another important and surprising outcome of our study is that recombinant strain can grow efficiently in Luria-Bertani Broth (LB), which costs less than R2YE medium. In the light of this information, it can be concluded that after optimization studies (carbon source, pH, temperature etc.) the recombinant strain has a high potential to be a strong candidate for industrial-grade lactic acid production using high-volume fermenters. Streptomyces were known for their secondary metabolite production in biotechnology sector, this study is the first one showing that this microorganism has also potential for the production of primary metabolites.

The growth conditions (carbon source, pH, temperature etc.) of the recombinant strain (S. coelicolor A3(2)+pRA^{str}) can be optimized for high lactic acid production in industrial scale. In addition, its lactic acid production capasity can be further increased through metabolic engineering approaches. Moreover, it is possible that the method could be applied to other Streptomyces strains and trigger the production of some metabolites that may be of industrial importance.

What is claimed is:

1. A method to trigger a lactic acid production in Streptomyces coelicolor A3(2), comprising the following process steps:
amplifying Streptomycin resistance gene (aadA) by a polymerase chain reaction (PCR) using pIJ778 plasmid as a template,
cloning the Streptomycin resistance gene (aadA) into an integrative and conjugative pRA plasmid to obtain a recombinant pRA^{str} plasmid,
transferring the recombinant pRA^{str} plasmid into Escherichia coli ET12567/pUZ8002 strain to obtain E. coli ET12567 /pUZ8002+pRA^{str}
performing [[the]]a conjugation between the Streptomyces coelicolor A3(2) and the E. coli ET12567/pUZ8002+pRA^{str}, and
obtaining a new recombinant cell (SCO+pRA^{str}).

2. The method according to claim 1, wherein a multicopy pSPG vector is allowed to be used instead of the integrative and conjugative pRA plasmid for cloning the Streptomycin resistance gene (aadA).

3. The method according to claim 1, wherein a pSPG^{str} recombinant plasmid is allowed to be transferred into the Escherichia coli ET12567/pUZ8002 strain.
